(19) United States
(12) Patent Application Publication
(10) Pub. No.: US 2026/0034325 A1
(43) Pub. Date: Feb. 5, 2026

(54) AN APPARATUS FOR ADMINISTRING GAS TO A PATIENT

(71) Applicant: MEDCLAIR AB, Stockholm (SE)

(72) Inventors: István SZABÓ, Boda Kyrkby (SE); Jerker SUNDLING, Borlänge (SE); Olesya NIKONOVA, Uppsala (SE)

(73) Assignee: MEDCLAIR AB, Stockholm (SE)

(21) Appl. No.: 18/997,311

(22) PCT Filed: Jul. 5, 2023
(86) PCT No.: PCT/SE2023/050708

§ 371 Date: Jan. 21, 2025

(30) Foreign Application Priority Data
Jul. 18, 2022 (SE) 2250916-0

(51) Int. Cl. A61M 16/06; A61M 16/10; A61M 16/12

(52) U.S. Cl. CPC A61M 16/06; A61M 16/1005; A61M 16/12; A61M 2202/0283

(57) ABSTRACT

An apparatus for administering gas to a patient, wherein the apparatus comprises a mask connectable to a coupler connectable to a gas source. The mask comprises a shell. The shell forms a first opening for receiving at least a portion of a mask member configured to fit over the nose and mouth of the patient for the administration of gas to the patient. The mask member is connectable to the coupler for providing gas to the mask member from the coupler. The shell is configured to house at least a portion of the mask member. The shell is configured to capture gas escaping from the mask member. The shell comprises one or more walls defining the first opening. The wall forms one or more second openings.

### TECHNICAL FIELD

Aspects of the present invention relate to an apparatus for administering gas to a patient. The apparatus comprises a mask connectable to a coupler connectable to a gas source.

### BACKGROUND

Nitrous oxide, also known as laughing gas, is used in the medical field for pain relief. In general, mixtures of nitrous oxide (N₂O) and oxygen (O₂) are used. For example, nitrous oxide may be used in the fields of surgery, dental care and maternity care during delivery because of the anaesthetic and analgesic effects of nitrous oxide on a patient. In general, nitrous oxide is administered to the patient via a mask fitted over the nose and/or mouth of the patent. In general, the choice of a suitable mask size is important due to possible leakage of gas between the mask and the patient's face. In general, the choice of the mask size will depend on the patient's face or nose size. For some conventional solutions, the mask may include an inner mask and an outer mask. The inner mask is fitted over the nose and/or mouth of the patient for the administration of nitrous oxide to the patient while the outer mask captures nitrous oxide leaking from the inner mask. Other gases may also be administered to a patient for various medical reasons.

### SUMMARY

The inventors of the present invention have found drawbacks in conventional solutions for masks used for the administration of gas to a patient. For example, some conventional masks for the administration of gas to a patient are not sufficiently efficient. For example, some conventional solutions for the administration of gas to a patient do not capture gas escaping from the mask in a sufficiently efficient manner.

An object of the invention is to provide a solution which mitigates or solves drawbacks and problems of conventional solutions.

The above and further objects are solved by the subject matter of the independent claims. Further advantageous embodiments of the invention can be found in the dependent claims.

According to a first aspect of the invention, the above mentioned and other objects are achieved with an apparatus for administering gas, or a gas mixture, to a patient, wherein the apparatus comprises a mask connectable to a coupler connectable to a gas source,
wherein the mask comprises a shell,
wherein the shell forms a first opening for receiving at least a portion of a mask member configured to fit over the nose and mouth of the patient for the administration of gas to the patient, the mask member being connectable to the coupler for providing gas to the mask member from the coupler,
wherein the shell is configured to house at least a portion of the mask member,
wherein the shell is configured to capture gas escaping from the mask member,
wherein the shell comprises one or more walls defining the first opening, and
wherein the wall forms one or more second openings.

An advantage of the apparatus according to the first aspect is that pressure relief is provided by the second opening when the shell and mask member are applied to the patient. An advantage of the apparatus according to the first aspect is that because of the one or more second openings a more even flow of the captured gas in the shell is provided compared to conventional solutions. An advantage of the apparatus according to the first aspect is that less turbulence in the flow of the captured gas in the shell is provided in relation to conventional solutions. An advantage of the apparatus according to the first aspect is that a lower flow rate of the captured gas, for example in the range of 20-25 l/min, or even lower, in the shell, so as to evacuate the captured gas, is required in relation to conventional solutions. In view of one or more of the above-mentioned advantages, another advantage of the apparatus according to the first aspect is that the noise produced by the apparatus when in use is reduced in relation to conventional solutions. Thus, conventional solutions require high air flow in the shell, leading to high noise exposure. An advantage of the apparatus according to the first aspect is that an improved mask used for the administration of gas to a patient is provided. An advantage of the apparatus according to the first aspect is that the administration of gas to a patient is improved.

An advantage of the apparatus according to the first aspect is that a gas-administration mask that is less complex in structure in relation to conventional solutions is provided. An advantage of the apparatus according to the first aspect is that the production of a mask used for the administration of gas to a patient is facilitated and improved.

An advantage of the apparatus according to the first aspect is that the task to prepare the mask for the next patient is facilitated and improved in relation to conventional solutions. For example, the task to prepare the mask for the next patient may involve assembling the mask and/or the apparatus. For example, the task to prepare the mask for the next patient may be performed by a user, such as a member of the staff. An advantage of the apparatus according to the first aspect is that the user-friendliness of the mask is improved.

For some embodiments, one or more of the shell and mask member may be made of a material comprising or consisting of a polymer or a polymer composite. However, other materials are possible. For some embodiments, the gas or gas mixture may comprise nitrous oxide (N₂O), also known as laughing gas. For some embodiments, the gas or gas mixture may comprise or consist of nitrous oxide and oxygen (O₂). However, other gases or gas mixtures are possible.

According to an advantageous embodiment of the apparatus according to the first aspect, the second opening is configured for pressure relief when the shell and mask member are applied to the patient. An advantage of this embodiment is that the mask used for the administration of gas to a patient is further improved. An advantage of this embodiment is that that the administration of gas to a patient is further improved.

According to an advantageous embodiment of the apparatus according to the first aspect, the first opening is configured to receive the mask member, wherein the shell is configured to house the mask member. An advantage of this embodiment is that the mask used for the administration of gas to a patient is further improved. An advantage of this embodiment is that that the administration of gas to a patient is further improved.

According to an advantageous embodiment of the apparatus according to the first aspect, the second opening opens in a direction parallel to the wall. An advantage of this embodiment is that dust, fibres and other small-sized materials are efficiently prevented from entering the shell through the second opening without any substantial blockage of the second opening. An advantage of this embodiment is that the mask used for the administration of gas to a patient is further improved. An advantage of this embodiment is that the administration of gas to a patient is further improved.

According to an advantageous embodiment of the apparatus according to the first aspect, the wall comprises one or more barriers at least partly surrounding the second opening. An advantage of this embodiment is that dust, fibres and other small-sized materials are efficiently prevented from entering the shell through the second opening. An advantage of this embodiment is that the mask used for the administration of gas to a patient is further improved. An advantage of this embodiment is that the administration of gas to a patient is further improved.

According to an advantageous embodiment of the apparatus according to the first aspect, the wall comprises two barriers at least partly surrounding the second opening.

According to an advantageous embodiment of the apparatus according to the first aspect, the two barriers partly overlap one another. An advantage of this embodiment is that dust, fibres and other small-sized materials are efficiently prevented from entering the shell through the second opening without any substantial blockage of the second opening. An advantage of this embodiment is that the mask used for the administration of gas to a patient is further improved. An advantage of this embodiment is that the administration of gas to a patient is further improved.

According to an advantageous embodiment of the apparatus according to the first aspect, the barrier is angled in relation to the remainder of the wall. An advantage of this embodiment is that dust, fibres and other small-sized materials are efficiently prevented from entering the shell through the second opening without any substantial blockage of the second opening. An advantage of this embodiment is that the mask used for the administration of gas to a patient is further improved. An advantage of this embodiment is that the administration of gas to a patient is further improved.

According to an advantageous embodiment of the apparatus according to the first aspect, the barrier is convex. An advantage of this embodiment is that dust, fibres and other small-sized materials are efficiently prevented from entering the shell through the second opening without any substantial blockage of the second opening. An advantage of this embodiment is that the mask used for the administration of gas to a patient is further improved. An advantage of this embodiment is that the administration of gas to a patient is further improved.

According to an advantageous embodiment of the apparatus according to the first aspect, the barrier has a curved border to the remainder of the wall. An advantage of this embodiment is that an efficient and resistant barrier is provided. An advantage of this embodiment is that the mask used for the administration of gas to a patient is further improved. An advantage of this embodiment is that the administration of gas to a patient is further improved.

According to an advantageous embodiment of the apparatus according to the first aspect, the second opening is adjacent to the first opening. An advantage of this embodiment is that an even more even flow of the captured gas in the shell is attained. An advantage of this embodiment is that even less turbulence in the flow of the captured gas in the shell is provided. An advantage of this embodiment is that an even lower flow rate of the captured gas in the shell is required to evacuate the captured gas. An advantage of this embodiment is that the noise produced by the apparatus when in use is further reduced. An advantage of this embodiment is that a further improved mask used for the administration of gas to a patient is provided. An advantage of this embodiment is that the administration of gas to a patient is further improved.

According to an advantageous embodiment of the apparatus according to the first aspect, the wall forms two or more second openings. An advantage of this embodiment is that an even more even flow of the captured gas in the shell is attained. An advantage of this embodiment is that even less turbulence in the flow of the captured gas in the shell is provided. An advantage of this embodiment is that an even lower flow rate of the captured gas in the shell is required to evacuate the captured gas. An advantage of this embodiment is that the noise produced by the apparatus when in use is further reduced. An advantage of this embodiment is that a further improved mask used for the administration of gas to a patient is provided. An advantage of this embodiment is that the administration of gas to a patient is further improved. For some embodiments, the wall forms four or more second openings. For some embodiments, the wall forms six or more second openings.

According to an advantageous embodiment of the apparatus according to the first aspect, the shell comprises a first seat for the nose of the patient and a second seat for the chin of the patient,
wherein the shell comprises a first lateral portion and a second lateral portion,
wherein the first lateral portion and the second lateral portion are joined at least in the first and second seats, and
wherein each one of the first and second lateral portions forms one or more of the second openings.

By way of this embodiment, the second openings are evenly distributed around the nose and mouth of the patient. An advantage of this embodiment is that an even more even flow of the captured gas in the shell is attained. An advantage of this embodiment is that even less turbulence in the flow of the captured gas in the shell is provided. An advantage of this embodiment is that an even lower flow rate of the captured gas in the shell is required to evacuate the captured gas. An advantage of this embodiment is that the noise produced by the apparatus when in use is further reduced. An advantage of this embodiment is that a further improved mask used for the administration of gas to a patient is provided. An advantage of this embodiment is that the administration of gas to a patient is further improved.

According to an advantageous embodiment of the apparatus according to the first aspect, the shell is configured to hold the mask member. An advantage of this embodiment is that a gas-administration mask that is even less complex in structure is provided. An advantage of this embodiment is that the task to prepare the mask for the next patient is further facilitated and improved. An advantage of this embodiment is that the user-friendliness of the mask is further improved. An advantage of this embodiment is that a further improved mask used for the administration of gas to a patient is provided. An advantage of this embodiment is that the administration of gas to a patient is further improved.

According to an advantageous embodiment of the apparatus according to the first aspect, the shell is configured to hold the mask member such that a gap is formed between the shell and the mask member. An advantage of this embodiment is that an even more even flow of the captured gas in the shell is attained. An advantage of this embodiment is that even less turbulence in the flow of the captured gas in the shell is provided. An advantage of this embodiment is that an even lower flow rate of the captured gas in the shell is required to evacuate the captured gas. An advantage of this embodiment is that the noise produced by the apparatus when in use is further reduced. An advantage of this embodiment is that a further improved mask used for the administration of gas to a patient is provided. An advantage of this embodiment is that the administration of gas to a patient is further improved.

According to an advantageous embodiment of the apparatus according to the first aspect, the shell comprises a border at least partly surrounding the first opening, wherein the wall of the shell is made of a first material while the border of the shell is made of a second material different form the first material. An advantage of this embodiment is that an even more even flow of the captured gas in the shell is attained. An advantage of this embodiment is that even less turbulence in the flow of the captured gas in the shell is provided. An advantage of this embodiment is that an even lower flow rate of the captured gas in the shell is required to evacuate the captured gas. An advantage of this embodiment is that the noise produced by the apparatus when in use is further reduced. An advantage of this embodiment is that a further improved mask used for the administration of gas to a patient is provided. An advantage of this embodiment is that the administration of gas to a patient is further improved.

According to an advantageous embodiment of the apparatus according to the first aspect, the second material is softer than the first material. An advantage of this embodiment is that an even more even flow of the captured gas in the shell is attained. An advantage of this embodiment is that even less turbulence in the flow of the captured gas in the shell is provided. An advantage of this embodiment is that an even lower flow rate of the captured gas in the shell is required to evacuate the captured gas. An advantage of this embodiment is that the noise produced by the apparatus when in use is further reduced. An advantage of this embodiment is that a further improved mask used for the administration of gas to a patient is provided. An advantage of this embodiment is that the administration of gas to a patient is further improved.

According to an advantageous embodiment of the apparatus according to the first aspect, when housing at least a portion of the mask member the shell is configured to fit over the nose and mouth of the patient during the administration of gas to the patient. An advantage of this embodiment is that even more gas escaped from the mask member can be captured, and that uncontrolled gas release from the mask member to the environment is further reduced or minimized. An advantage of this embodiment is that an even more even flow of the captured gas in the shell is attained. An advantage of this embodiment is that even less turbulence in the flow of the captured gas in the shell is provided. An advantage of this embodiment is that an even lower flow rate of the captured gas in the shell is required to evacuate the captured gas. An advantage of this embodiment is that the noise produced by the apparatus when in use is further reduced. An advantage of this embodiment is that a further improved mask used for the administration of gas to a patient is provided. An advantage of this embodiment is that the administration of gas to a patient is further improved.

According to an advantageous embodiment of the apparatus according to the first aspect, the shell comprises a border surrounding the first opening of the shell, wherein the border of the shell is configured to abut against the patient during the administration of gas to the patient. An advantage of this embodiment is that the mask used for the administration of gas to a patient is further improved. An advantage of this embodiment is that the administration of gas to a patient is further improved. An advantage of this embodiment is that even more gas escaped from the mask member can be captured, and that uncontrolled gas release from the mask member to the environment is further reduced or minimized.

According to an advantageous embodiment of the apparatus according to the first aspect, the mask member forms an opening for surrounding the nose and mouth of the patient for the administration of gas to the patient,
wherein the mask member comprises a border surrounding the opening of the mask member,
wherein the border of the mask member is configured to abut against the patient during the administration of gas to the patient, and
wherein the border of the shell and the border of the mask member are configured to be substantially in the same plane when applied to the patient. An advantage of this embodiment is that the mask used for the administration of gas to a patient is further improved. An advantage of this embodiment is that the administration of gas to a patient is further improved. An advantage of this embodiment is that even more gas escaped from the mask member can be captured, and that uncontrolled gas release from the mask member to the environment is further reduced or minimized.

According to an advantageous embodiment of the apparatus according to the first aspect, the border of the shell is resilient. An advantage of this embodiment is that the mask used for the administration of gas to a patient is further improved. An advantage of this embodiment is that the administration of gas to a patient is further improved. An advantage of this embodiment is that even more gas escaped from the mask member can be captured, and that uncontrolled gas release from the mask member to the environment is further reduced or minimized. For example, an advantage of this embodiment is that the partial sealing of the interior of the shell from the exterior of the shell when applied to the patient is improved.

According to an advantageous embodiment of the apparatus according to the first aspect, the shell is reusable while the mask member is disposable. An advantage of this embodiment is that the mask used for the administration of gas to a patient is further improved.

According to an advantageous embodiment of the apparatus according to the first aspect, the shell is attachable to the mask member. An advantage of this embodiment is that the mask used for the administration of gas to a patient is further improved. An advantage of this embodiment is that the user-friendliness of the mask is further improved. An advantage of this embodiment is that the task to prepare the mask for the next patient is further facilitated and further improved. An advantage of this embodiment is that the administration of gas to a patient is further improved.

According to an advantageous embodiment of the apparatus according to the first aspect, the shell is detachably attachable to the mask member and easily detachable from the mask member. An advantage of this embodiment is that the mask used for the administration of gas to a patient is further improved. An advantage of this embodiment is that the user-friendliness of the mask is further improved. An advantage of this embodiment is that the task to prepare the mask for the next patient is further facilitated and further improved. An advantage of this embodiment is that the administration of gas to a patient is further improved.

According to an advantageous embodiment of the apparatus according to the first aspect, the shell is cup-shaped. An advantage of this embodiment is that the mask used for the administration of gas to a patient is further improved. An advantage of this embodiment is that the administration of gas to a patient is further improved.

According to an advantageous embodiment of the apparatus according to the first aspect, the shell comprises one or more outlets for guiding away gas escaped from the mask member and captured by the shell. An advantage of this embodiment is that the mask used for the administration of gas to a patient is further improved. An advantage of this embodiment is that the administration of gas to a patient is further improved.

According to an advantageous embodiment of the apparatus according to the first aspect, the apparatus comprises the coupler connectable to the gas source.

According to an advantageous embodiment of the apparatus according to the first aspect, the coupler is connectable to a cylinder configured to contain a gas or a gas mixture. An advantage of this embodiment is that the administration of gas to a patient is further improved.

According to an advantageous embodiment of the apparatus according to the first aspect, the coupler is configured to feed a gas mixture to the mask member, the gas mixture comprising or consisting of nitrous oxide and oxygen.

According to an advantageous embodiment of the apparatus according to the first aspect, the coupler comprises one or more gas valves. An advantage of this embodiment is that the administration of gas to a patient is further improved.

According to an advantageous embodiment of the apparatus according to the first aspect, when assembled the shell is movable in relation to the mask member in a resilient manner. An advantage of this embodiment is that the mask used for the administration of gas to a patient is further improved. An advantage of this embodiment is that the administration of gas to a patient is further improved. An advantage of this embodiment is that even more gas escaped from the mask member can be captured, and that uncontrolled gas release from the mask member to the environment is further reduced or minimized.

According to an advantageous embodiment of the apparatus according to the first aspect, the apparatus comprises the mask member.

According to a second aspect of the invention, the above mentioned and other objects are achieved with an arrangement for administering gas, or a gas mixture, to a patient, wherein the arrangement comprises a mask connectable to a coupler connectable to a gas source,
wherein the mask comprises a shell configured to house at least a portion of a mask member configured to fit over the nose and mouth of the patient for the administration of gas to the patient, the mask member being connectable to the coupler for providing gas to the mask member from the coupler,
wherein the shell is configured to capture gas escaping from the mask member, and
wherein when housing at least a portion of the mask member the shell is configured to fit over the nose and mouth of the patient during the administration of gas to the patient.

An advantage of the arrangement according to the second aspect is that more gas escaped from the mask member can be captured in relation to conventional solutions, and that uncontrolled gas release from the mask member to the environment is reduced or minimized. An advantage of the arrangement according to the second aspect is that an improved mask used for the administration of gas to a patient is provided. An advantage of the arrangement according to the second aspect is that the administration of gas to a patient is improved.

According to an advantageous embodiment of the arrangement according to the second aspect, the arrangement comprises the mask member.

According to an advantageous embodiment of the arrangement according to the second aspect, the mask member forms an opening for surrounding the nose and mouth of the patient for the administration of gas to the patient,
wherein the mask member comprises a border surrounding the opening of the mask member,
wherein the shell forms a first opening for receiving at least a portion of the mask member,
wherein the shell comprises a border surrounding the first opening of the shell,
wherein the border of the mask member is configured to abut against the patient during the administration of gas to the patient, and
wherein the border of the shell is configured to abut against the patient during the administration of gas to the patient.

An advantage of this embodiment is that the mask used for the administration of gas to a patient is further improved. An advantage of this embodiment is that that the administration of gas to a patient is further improved.

According to an advantageous embodiment of the arrangement according to the second aspect, the border of the shell is configured to substantially seal the interior of the shell from the exterior of the shell when applied to the patient. An advantage of this embodiment is that the mask used for the administration of gas to a patient is further improved. An advantage of this embodiment is that that the administration of gas to a patient is further improved.

According to an advantageous embodiment of the arrangement according to the second aspect, the border of the shell and the border of the mask member are configured to be substantially in the same plane when applied to the patient. An advantage of this embodiment is that the mask used for the administration of gas to a patient is further improved. An advantage of this embodiment is that that the administration of gas to a patient is further improved.

According to an advantageous embodiment of the arrangement according to the second aspect, one or more of the borders of the mask member and shell is/are resilient. An advantage of this embodiment is that the mask used for the administration of gas to a patient is further improved. An advantage of this embodiment is that that the administration of gas to a patient is further improved.

According to an advantageous embodiment of the arrangement according to the second aspect, the shell comprises one or more walls, wherein the wall forms one or more second openings or recesses. An advantage of this embodiment is that the mask used for the administration of gas to a patient is further improved. An advantage of this embodiment is that that the administration of gas to a patient is further improved.

According to an advantageous embodiment of the arrangement according to the second aspect, the mask member and the shell are movable in relation to one another in a resilient manner. An advantage of this embodiment is that the mask used for the administration of gas to a patient is further improved. An advantage of this embodiment is that that the administration of gas to a patient is further improved.

The above-mentioned features and embodiments of the apparatus and arrangement may be combined in various possible ways providing further advantageous embodiments.

Further advantageous embodiments of the apparatus according to the first aspect and of the arrangement according to the second aspect and further advantages with the embodiments of the present invention emerge from the detailed description of embodiments.

### BRIEF DESCRIPTION OF THE DRAWINGS

Embodiments of the invention will now be illustrated, for exemplary purposes, in more detail by way of embodiments and with reference to the enclosed drawings, where similar references are used for similar parts, in which:

FIG. 1 is a schematic perspective front view of an embodiment of the apparatus according to the first aspect of the invention and an embodiment of the arrangement according to the second aspect of the invention;

FIG. 2 is a schematic side view of the apparatus of FIG. 1;

FIG. 3 is a schematic top view of the apparatus of FIG. 1;

FIG. 4 is a schematic bottom view of the apparatus of FIG. 1;

FIG. 5 is a schematic rear view of the apparatus of FIG. 1;

FIG. 6 is a schematic front view of the apparatus of FIG. 1;

FIG. 7 schematically illustrates a cross-section of the apparatus along A-A in FIG. 6;

FIG. 8 schematically illustrates a cross-section of the apparatus along B-B in FIG. 6;

FIG. 9 is a schematic perspective front view of the apparatus and of the arrangement of FIG. 1 provided with a mask member;

FIG. 10 is a schematic side view of the apparatus of FIG. 9;

FIG. 11 is a schematic top view of the apparatus of FIG. 9;

FIG. 12 is a schematic bottom view of the apparatus of FIG. 9;

FIG. 13 is a schematic rear view of the apparatus of FIG. 9;

FIG. 14 is a schematic front view of the apparatus of FIG. 4;

FIG. 15 schematically illustrates a cross-section of the apparatus along C-C in FIG. 14;

FIG. 16 schematically illustrates a cross-section of the apparatus along D-D in FIG. 14;

FIG. 17 is a schematic perspective rear view of the apparatus and of the arrangement of FIG. 1;

FIG. 18 is an enlargement of a portion H of the apparatus of FIG. 17;

FIG. 19 is a schematic side view of the apparatus of FIG. 17;

FIG. 20 schematically illustrates a section of the apparatus along E-E in FIG. 19; and

FIG. 21 schematically illustrates a section of the apparatus along F-F in FIG. 19.

### DETAILED DESCRIPTION

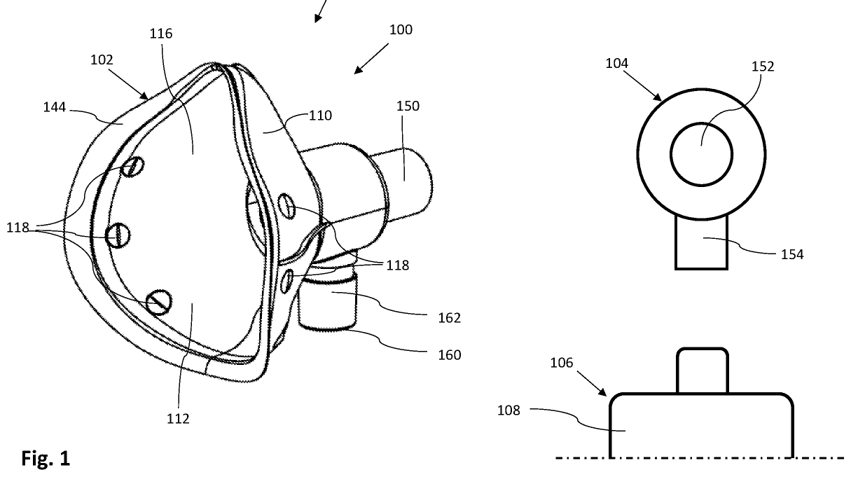

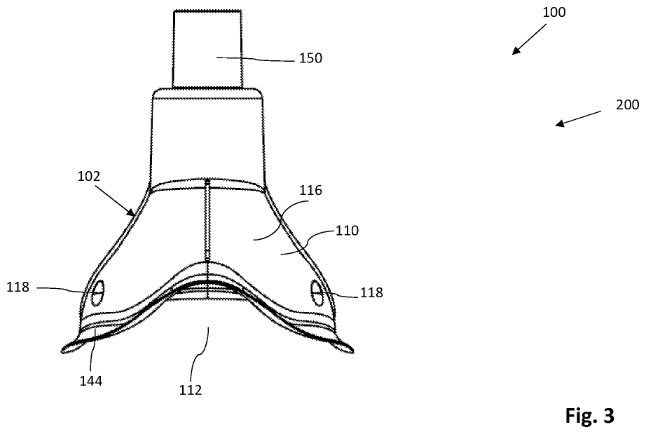

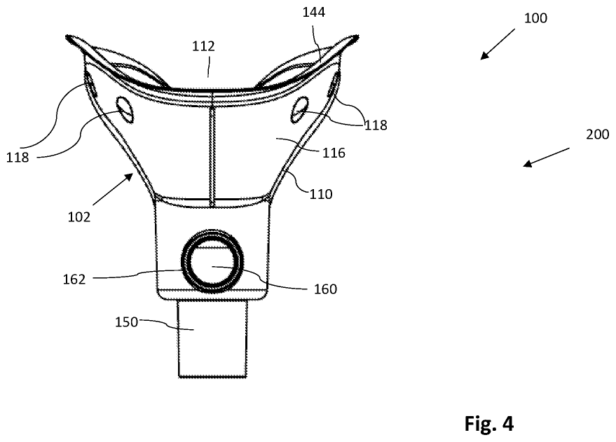

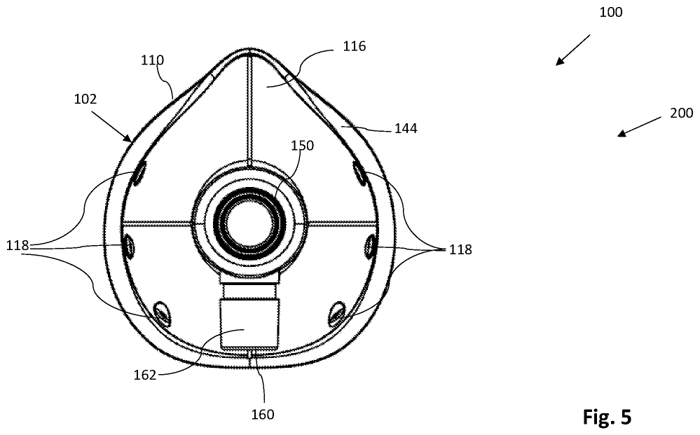

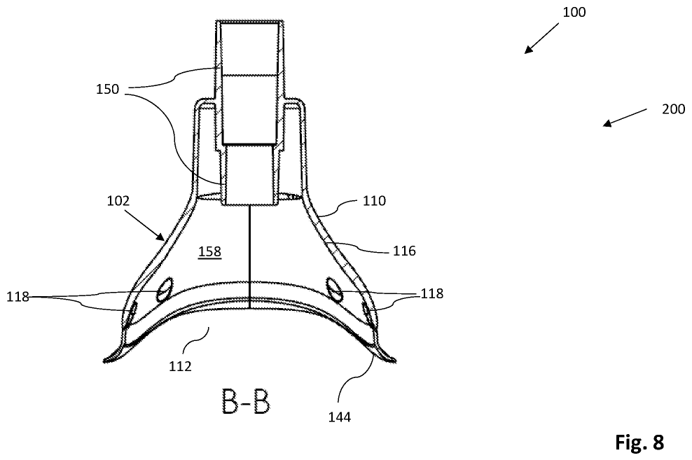

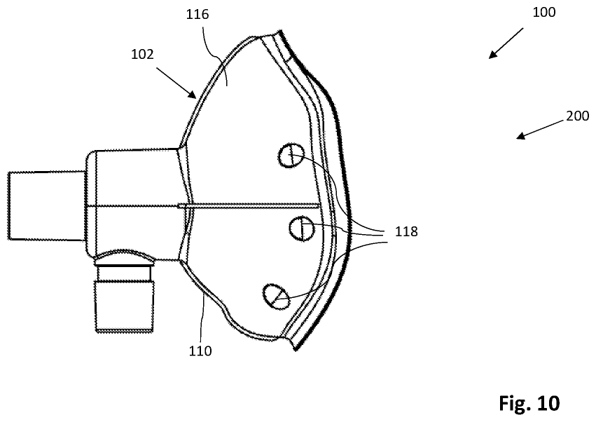

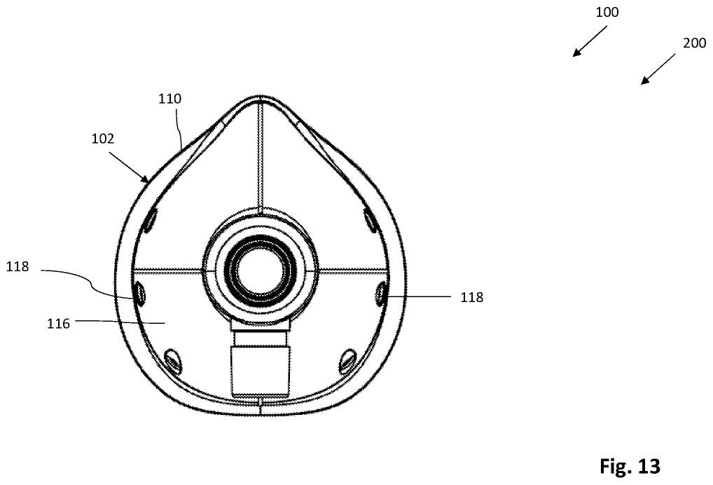

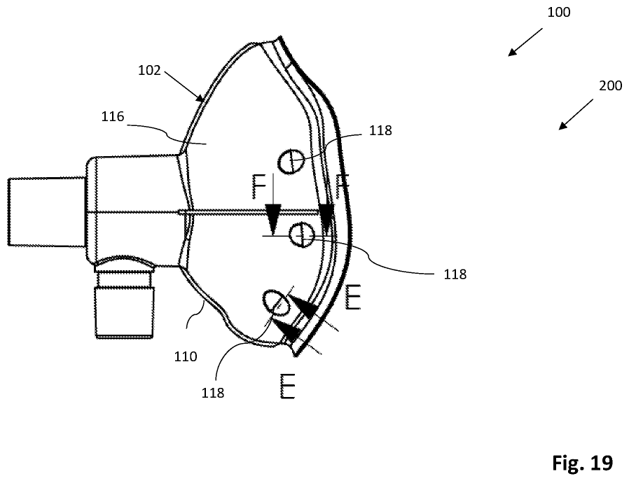

With reference to FIGS. 1 to 21, an embodiment of the apparatus 100 and aspects of embodiments of the apparatus 100 for administering gas, or a gas mixture, to a patient according to the first aspect of the invention are schematically illustrated. Further, an embodiment of the arrangement 200 and aspects of embodiments of the arrangement 200 for administering gas, or a gas mixture, to a patient according to the second aspect of the invention are schematically illustrated. For some embodiments, the gas or gas mixture may comprise nitrous oxide (N₂O), also known as laughing gas. For some embodiments, the gas or gas mixture may comprise or consist of nitrous oxide and oxygen (O₂). However, other gases or gas mixtures are possible.

With reference to FIG. 1, the apparatus 100 includes a mask 102 connectable to a coupler 104. The coupler 104 is connectable to a gas source 106, for example via one or more tubes or lines, or directly connectable. For some embodiments, the gas source 106 may comprise a cylinder 108 configured to contain a gas or a gas mixture, such as nitrous oxide and oxygen. Thus, the coupler 104 may be connectable to the cylinder 108 configured to contain a gas or a gas mixture.

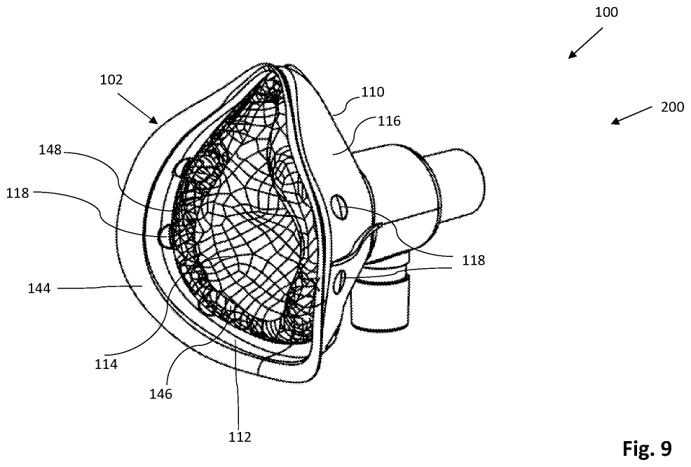

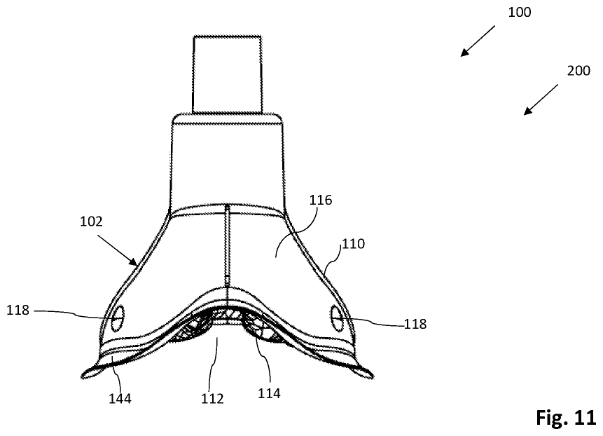

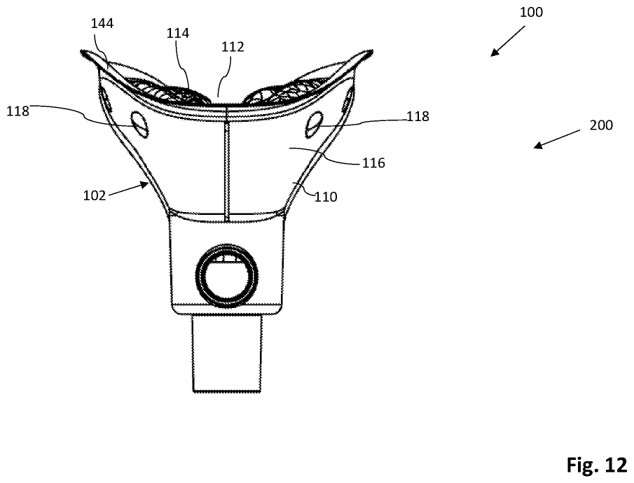

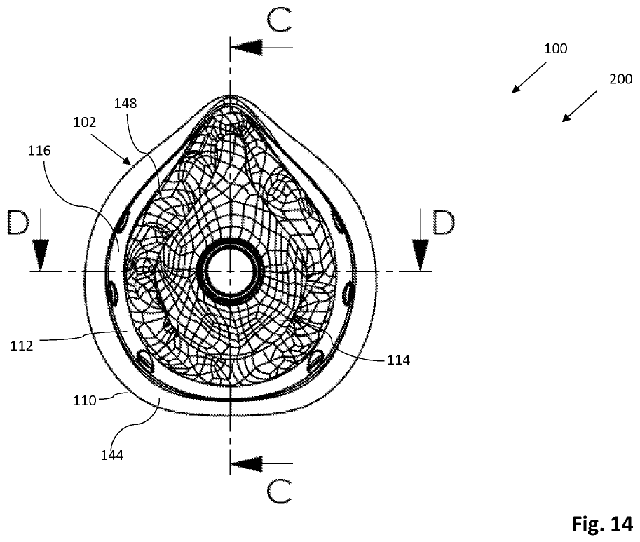

With reference to FIGS. 1 to 17, the mask 102 includes a shell 110. The shell 110 forms a first opening 112 for receiving at least a portion of a mask member 114. The mask member 114 is visible in FIGS. 9 and 14 and also partly visible in FIGS. 11 and 12. The mask member 114 is configured to fit over the nose and mouth of the patient for the administration of gas to the patient. The mask member 114 is connectable to the coupler 104 for providing gas to the mask member 114 from the coupler 104. The first opening 112 of the shell 110 may be described to be configured to receive at least a portion of a mask member 114. The shell 110 is configured to house at least a portion of the mask member 114. The shell 110 is configured to capture gas escaping from the mask member 114. The shell 110 includes one or more walls 116 defining the first opening 112. The wall 116 forms one or more second openings 118, or through-holes. For some embodiments, the second opening 118 may be described to be configured for pressure relief when the shell 110 and the mask member 114 are applied to the patient. For some embodiments, it may be defined that the second opening 118 is spaced apart from the first opening 112. For some embodiments, the second opening 118 may be adjacent to the first opening 112. For some embodiments, the shell 110 may be described to be cup-shaped. The mask member 114 may be described to be cup-shaped.

With reference to FIGS. 1 to 17, for some embodiments, the first opening 112 of the shell 110 may be configured to receive the mask member 114, such as the entire mask member 114. For some embodiments, the shell 110 may be configured to house the mask member 114, such as the entire mask member 114.

With reference to FIGS. 1 to 17, for some embodiments, the wall 116 of the shell 110 may form two or more second openings 118. For some embodiments, the wall 116 of the shell 110 may form four or more second openings 118. For some embodiments, the wall 116 of the shell 110 may form six or more second openings 118. In the embodiment illustrated in FIGS. 1 to 17, the wall 116 of the shell 110 forms six second openings 118. However, it is to be understood that for some embodiments the wall 116 may form fewer or more than six second openings 118.

For some embodiments, the first opening 112 of the shell 110 may be more than 50 to 70 times, such as more than 100 times, larger than the second opening 118 of the shell 110.

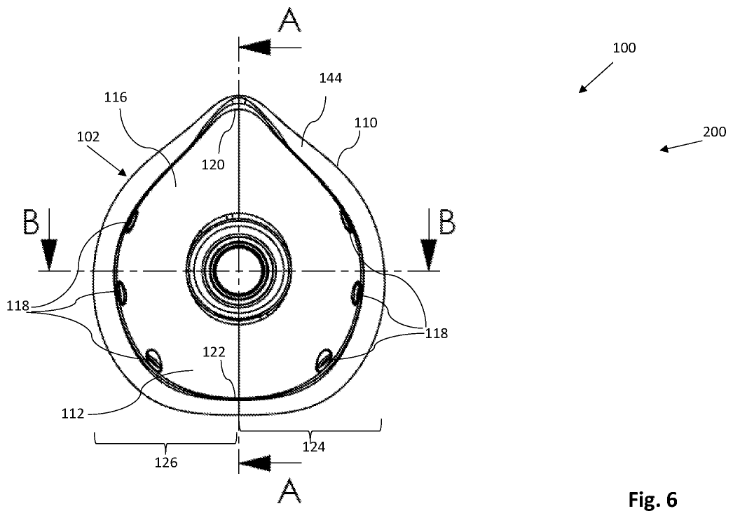

With reference to FIG. 6, for some embodiments, it may be defined that the shell 110 comprises, or forms, a first seat 120 for the nose of the patient and a second seat 122 for the chin of the patient. The shell 110 may include a first lateral portion 124 and a second lateral portion 126. The first lateral portion 124 and the second lateral portion 126 may be described to be joined at least in the first and second seats 120, 122. For some embodiments, each one 124, 126 of the first and second lateral portions 124, 126 forms one or more of the second openings 118, for example two or more of the second openings 118, for example three or more of the second openings 118. In the embodiment illustrated in FIGS. 1 to 17, each one 124, 126 of the first and second lateral portions 124, 126 forms three of the second openings 118. However, it is to be understood that for some embodiments each one 124, 126 of the first and second lateral portions 124, 126 may form fewer or more than three second openings 118. For some embodiments, it may be defined that the first and second lateral portions 124, 126 form the shell 110.

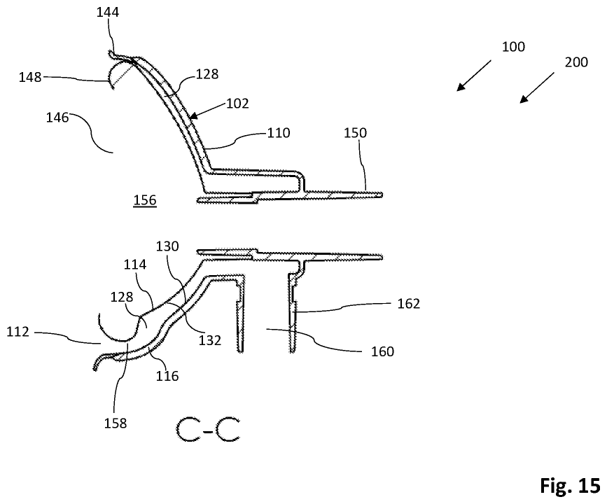

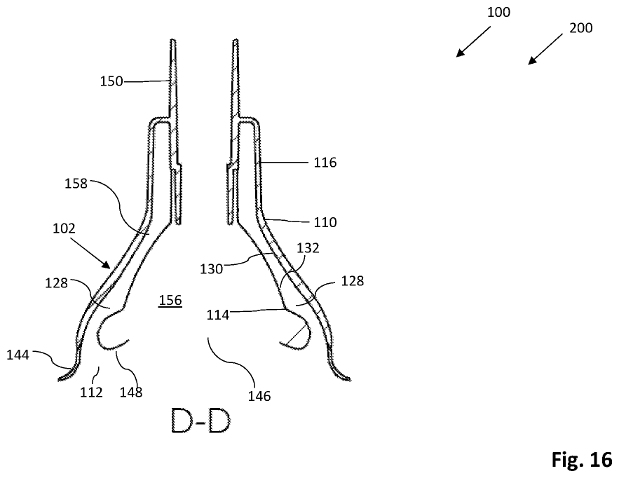

With reference to FIGS. 15 and 16, for some embodiments, the shell 110 may be configured to hold the mask member 114. The shell 110 may be configured to hold the mask member 114 such that a gap 128 is formed between the shell 110 and the mask member 114. More specially, said gap 128 may be formed between an inner surface 130 of the wall 116 of shell 110 and an outer surface 132 of the mask member 114. The gap 128 between the shell 110 and the mask member 114 may be configured to capture gas escaping from the mask member 114. For some embodiments, the shell 110 may be configured to hold the mask member 114 such that the mask member 114 is centrally positioned within the shell 110. For some embodiments, said gap 128 may have a width of about 4 to 7 mm, such as 5 to 6 mm. However, at some places, the gap 128 may be smaller, such as at the first seat 120 for the nose of the patient. For some embodiments, at some places, such as at the first seat 120 for the nose of the patient, the shell 110 may even abut against the mask member 114.

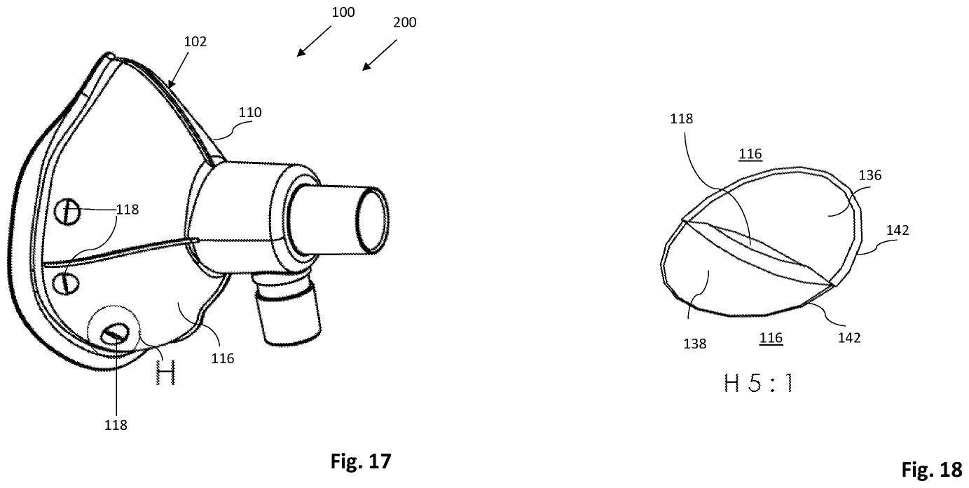

With reference to FIGS. 17 to 21, for some embodiments, the second opening 118 may open in a direction 134 parallel to the wall 116 of the shell 110 (see FIG. 21). For some embodiments, the wall 116 of the shell 110 may include, or may form, one or more barriers 136, 138 at least partly surrounding the second opening 118. The barrier 136, 138 may comprise a lip. For some embodiments, the wall 116 of the shell 110 may include two barriers 136, 138 at least partly surrounding the second opening 118, for example as illustrated in the embodiment in FIGS. 17 to 21. Thus, the wall 116 of the shell 110 may include two barriers 136, 138 for each second opening 118. For some embodiments, the two barriers 136, 138 may partly overlap one another, for example as illustrated in FIG. 21. It is to be understood that for some embodiments the one or more barriers 136, 138 may be excluded.

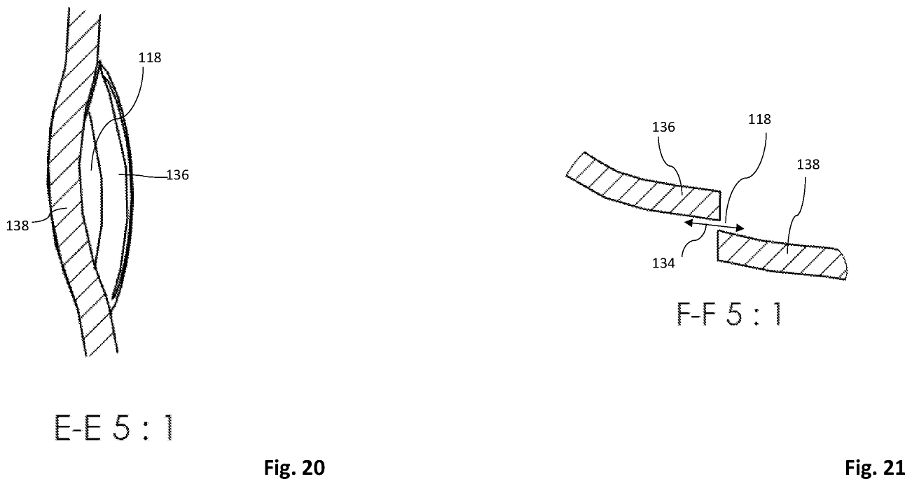

With reference to FIG. 20, the barrier 136, 138 may be angled in relation to the remainder of the wall 116 of the shell 110. For some embodiments, the barrier 136, 138 may be convex, or concave. For example, the barrier 136 may be outwardly convex in a direction away from the interior of the shell 110. For example, the barrier 138 may be inwardly convex in a direction toward the interior of the shell 110. For some embodiments, for the two barriers 136, 138 associated with the same second opening 118, one 136 of the two barriers 136, 138 may be outwardly convex while the other one 138 of the two barriers 136, 138 may be inwardly convex. With reference to FIGS. 17 and 18, the barrier 136, 138 may have a curved border 140, 142 to the remainder of the wall 116 of the shell 110.

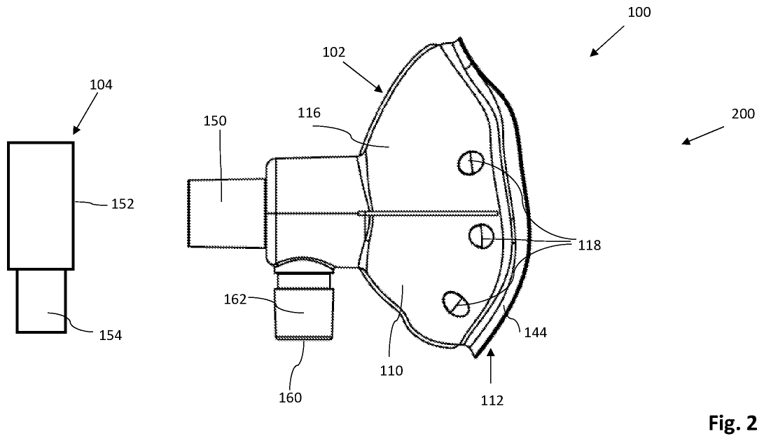

With reference to FIGS. 1 and 2, the shell 110 may be described to include a border 144 at least partly surrounding the first opening 112. For some embodiments, the wall 116 of the shell 110 may be made of a first material while the border 144 of the shell 110 may be made of a second material different form the first material. For some embodiments, the second material may be softer than the first material. For some embodiments, the border 144 of the shell 110 may be resilient. For some embodiments, one or more of the shell 110 and mask member 114 may be made of a material comprising or consisting of a polymer or a polymer composite. For example, the second material of the border 144 of the shell 110 may comprise or consist of silicone. For some embodiments, the shell 110, the wall 116 of the shell 110 and/or the first material of the wall 116 of the shell 110 may be transparent. However, other materials are possible. For some embodiments, the border 144 of the shell 110 may be thinner, or smaller, at the first seat 120 for the nose of the patient than outside the first seat 120.

With reference to FIGS. 9 to 16, for some embodiments, when housing at least a portion of the mask member 114, or the entire mask member 114, the shell 110 may be configured to fit over the nose and mouth of the patient during the administration of gas to the patient. When the shell 110 includes a border 144 surrounding the first opening 112 of the shell 110, the border 144 of the shell 110 may be configured to abut against the patient during the administration of gas to the patient. The mask member 114 may form an opening 146 for surrounding the nose and mouth of the patient for the administration of gas to the patient. The mask member 114 may include a border 148 surrounding the opening 146 of the mask member 114. The border 148 of the mask member 114 may be configured to abut against the patient during the administration of gas to the patient. The border 144 of the shell 110 and the border 148 of the mask member 114 may be configured to be substantially in the same plane when applied to the patient.

With reference to FIGS. 1 to 21, for some embodiments, the shell 110 may be reusable while the mask member 114 is disposable. The shell 110 may be configured to receive, house and/hold mask members 114 of different sizes, and the size of the mask member 114 may be adapted to the patient, such as to the shape and size of the face of the patient. The mask member 114 may be an off-the-shelf product. The shell 110 may be attachable to the mask member 114. The shell 110 may be detachably attachable to the mask member 114 and easily detachable from the mask member 114. For some embodiments, when assembled, the shell 110 may be movable in relation to the mask member 114 in a resilient manner. The resilience discussed above may be called mechanical resilience. For some embodiments, the apparatus 100 may include the mask member 114.

With reference to FIGS. 1 to 16, for some embodiments, the apparatus 100 may include the coupler 104 connectable to the gas source 106. The coupler 104 may be connectable to a cylinder 108 configured to contain a gas or a gas mixture. The coupler 104 may be configured to feed a gas mixture to the mask member 114, the gas mixture comprising or consisting of nitrous oxide and oxygen. The coupler 104 may include one or more gas valves (not shown).

With reference to FIGS. 1 to 16, for some embodiments, for some embodiments, the apparatus 100 may include a first tubular member 150. The first tubular member 150 may be described to form a tube. The first tubular member 150 may be described to be hollow. The first tubular member 150 may be configured to connect the mask 102 to the coupler 104 connectable to the gas source 106. The coupler 104 may be configured to feed gas, or a gas mixture, to the mask member 114 via the first tubular member 150. The coupler 104 may be configured to receive exhalation air from the patient via the first tubular member 150. The first tubular member 150 may be configured to connect the mask member 114 to the gas valve of the coupler 104 for providing gas to the mask member 114 from the coupler 104.

With reference to FIGS. 1 and 2, for some embodiments, the coupler 104 may form an opening 152, or passage, for receiving the first tubular member 150. For some embodiments, the coupler 104 and/or the opening 152 of the coupler 104 may be configured to hold the first tubular member 150. The coupler 104 may comprise an inlet and/or outlet member 154 configured for the connection to the gas source 106. As mentioned above, the gas source 106 may comprise a cylinder 108, such as a gas cylinder. Thus, for some embodiments, the coupler 104 may be connectable to the cylinder 108 via the inlet and/or outlet member 154. For some embodiments, the coupler 104 may be referred to as a coupling unit, a coupling housing, or a connector.

With reference to FIGS. 1 to 16, for some embodiments, the first tubular member 150 may be configured to attach the mask 102 to the coupler 104. For some embodiments, the first tubular member 150 may be configured to attach the shell 110 to the coupler 104. The first tubular member 150 may be configured to connect the mask member 114 to the coupler 104 for providing gas to the mask member 114 from the coupler 104, and thus to the patient when the mask member 114 is applied to the patient. The first tubular member 150 may be attachable to the mask member 114. The first tubular member 150 may be configured to be in physical contact with the mask member 114.

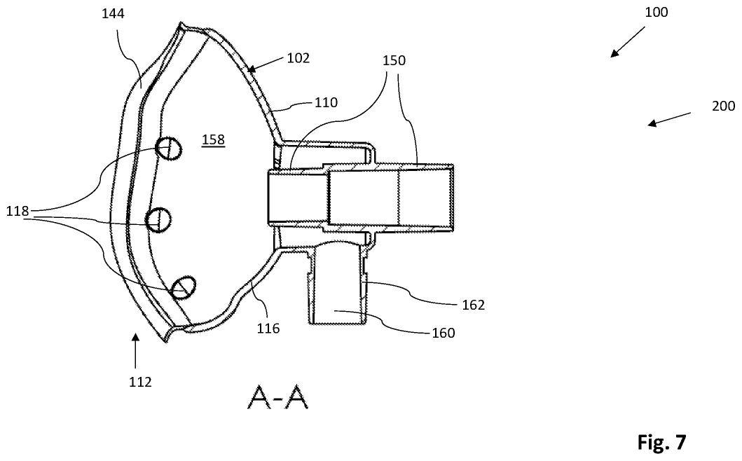

With reference to FIGS. 7, 8, 15 and 16, for some embodiments, it may be described that the mask member 114 forms, or defines, a space 156 configured to receive the nose and mouth of the patient. For some embodiments, it may be described that the shell 110 defines, or forms, a space 158 configured to receive and/or house at least one portion of the mask member 114. For some embodiments, the shell 110 may be described to capture gas escaping from the mask member 114 and not returning, or avoiding a return path, through the first tubular member 150. For some embodiments, it may be described that the first tubular member 150 is configured to connect the space 156 of the mask member 114 to the coupler 104 for providing gas to the space 156 defined by the mask member 114 from the coupler 104, and thus to the patient when the nose and mouth of the patient is/are received by the space 156 of the mask member 114. For some embodiments it may be described that the second opening 118 provides a passage, such as a flow/fluid passage, between the ambient outside the shell 110 and the space 158 defined by the shell 110 and/or the gap 128.

With reference to FIGS. 9 to 16, for some embodiments, the first tubular member 150 may be configured to hold the mask member 114. For some embodiments, the first tubular member 150 may be described to be detachable, or releasable, in relation to the mask member 114. The first tubular member 150 and the mask member 114 may be easily detachable in relation to, or from, one another. The first tubular member 150 may be attachable and/or detachable in relation to, or from, the mask member 114, for example by way of a locking arrangement comprising one or more of the group of:
a friction locking arrangement;
a positive locking arrangement;
a bayonet locking arrangement;
a snap-on locking arrangement;
a snap-in locking arrangement; and
a threaded locking arrangement.

With reference to FIGS. 1 to 16, for some embodiments, the first tubular member 150 may be integrally formed with the shell 110, which prevents leakage of gas in an efficient manner. However, for alternative embodiments, the first tubular member 150 may be attached, or attachable, to the shell 110 in other manners, for example by way of an adhesive, or by a locking arrangement, for example a locking arrangement of any one of the sorts mentioned above.

With reference to FIGS. 1 to 16, for some embodiments, the shell 110, or the apparatus 100, may include one or more outlets 160 for guiding away gas escaped from the mask member 114 and captured by the shell 110. The outlet 160 may be connectable, for example via one or more tubes or lines, or directly, to a gas collector, or a decomposition unit for the decomposition of the escaped gas, such escaped nitrous oxide. The decomposition unit may be mobile or central. The apparatus 100 may comprise a second tubular member 162 including, or forming, the outlet 160 for guiding away the escaped gas. The second tubular member 162 may be described to form a tube. The second tubular member 162 may be described to be hollow. The second tubular member 162 may be integrally formed with the shell 110, which prevents leakage of gas in an efficient manner. However, for alternative embodiments, the second tubular member 162 may be attached, or attachable, to the shell 110 in other manners, for example by way of an adhesive, or by a locking arrangement, for example a locking arrangement of any one of the sorts mentioned above. The outlet 160 and/or the second tubular member 162 may be connected to the gap 128 formed between the shell 110 and the mask member 114 and/or to the space 158 formed by the shell 110. The second tubular member 162 may be connectable, for example via one or more tubes or lines, to the gas collector, or the decomposition unit mentioned above.

For some embodiments, one or more of the first tubular member 150 and second tubular member 162 may be made of a material comprising or consisting of a polymer or a polymer composite. However, other materials are possible.

With reference to FIGS. 1 to 21, as mentioned above, also an embodiment of the arrangement 200 according to the second aspect is schematically illustrated. The arrangement comprises a mask 102 connectable to a coupler 104 connectable to a gas source 106. The mask 102 comprises a shell 110 configured to house at least a portion of a mask member 114 configured to fit over the nose and mouth of the patient for the administration of gas to the patient. The mask member 114 is connectable to the coupler 104 for providing gas to the mask member 114 from the coupler 104. The shell 110 is configured to capture gas escaping from the mask member 114. When housing at least a portion of the mask member 114, or the entire mask member 114, the shell 110 is configured to fit over the nose and mouth of the patient during the administration of gas to the patient. For some embodiments, the arrangement 200 comprises the mask member 114.

For some embodiments of the arrangement 200, the mask member 114 may form an opening 146 for surrounding the nose and mouth of the patient for the administration of gas to the patient, wherein the mask member 114 may include a border 148 surrounding the opening 146 of the mask member 116, wherein the shell 110 may form a first opening 112 for receiving at least a portion of the mask member 114, wherein the shell may include a border 144 surrounding the first opening 112 of the shell 110, wherein the border 148 of the mask member 114 may be configured to abut against the patient during the administration of gas to the patient, and wherein the border 144 of the shell 110 may be configured to abut against the patient during the administration of gas to the patient. For some embodiments of the arrangement 200, the border 144 of the shell 110 may be configured to substantially seal the interior of the shell 110 from the exterior of the shell 110 when applied to the patient. For some embodiments of the arrangement 200, the border 144 of the shell 110 and the border 148 of the mask member 144 may be configured to be substantially in the same plane when applied to the patient. For some embodiments of the arrangement 200, one or more of the borders 144, 148 of the mask member 114 and shell 110 may be resilient. For some embodiments of the arrangement 200, the shell 110 may include one or more walls 116, wherein the wall 116 may form one or more second openings 118 or one or more recesses. For some embodiments of the arrangement 200, the mask member 114 and the shell 110 may be movable in relation to one another in a resilient manner.

When an item is disclosed to be connected to another item in this disclosure, it may imply that the two items are fluidly connected to one another. When an item is disclosed to be connected to another item in this disclosure, it may imply that the two items are mechanically connected to one another. When an item is disclosed to be connected to another item in this disclosure, it may imply that the two items are both fluidly and mechanically connected to one another.

The present invention is not limited to the above-described embodiments. Instead, the present invention relates to, and encompasses all different embodiments being included within the scope of the appended independent claims.

1. An apparatus for administering gas to a patient, wherein the apparatus comprises a mask connectable to a coupler connectable to a gas source,
wherein the mask comprises a shell,
wherein the shell forms a first opening for receiving at least a portion of a mask member configured to fit over the nose and mouth of the patient for the administration of gas to the patient, the mask member being connectable to the coupler for providing gas to the mask member from the coupler,
wherein the shell is configured to house at least a portion of the mask member,
wherein the shell is configured to capture gas escaping from the mask member,
wherein the shell comprises one or more walls defining the first opening, and
wherein the wall forms one or more second openings.

2. The apparatus according to claim 1, wherein the second opening is configured for pressure relief when the shell and mask member are applied to the patient.

3. The apparatus according to claim 1, wherein the first opening is configured to receive the mask member, and
wherein the shell is configured to house the mask member.

4. The apparatus according to claim 1 to, wherein the second opening opens in a direction parallel to the wall.

5. The apparatus according to claim 1 to, wherein the wall comprises one or more barriers at least partly surrounding the second opening.

6. The apparatus according to claim 1, wherein the wall comprises two barriers at least partly surrounding the second opening.

7. The apparatus according to claim 6, wherein the two barriers partly overlap one another.

8. The apparatus according to claim 5, wherein the barrier is angled in relation to the remainder of the wall.

9. The apparatus according to claim 5, wherein the barrier is convex.

10. The apparatus according to claim 5, wherein the barrier has a curved border to the remainder of the wall.

11. (canceled)

12. The apparatus according to claim 1, wherein the wall forms two or more second openings.

13. (canceled)

14. (canceled)

15. The apparatus according to claim 1, wherein the shell is configured to hold the mask member such that a gap is formed between the shell and the mask member.

16. (canceled)

17. (canceled)

18. The apparatus according to claim 1, wherein when housing at least a portion of the mask member the shell is configured to fit over the nose and mouth of the patient during the administration of gas to the patient.

19. The apparatus according to claim 1, wherein the shell comprises a border surrounding the first opening of the shell, and
wherein the border of the shell is configured to abut against the patient during the administration of gas to the patient.

20. (canceled)

21. (canceled)

22. The apparatus according to claim 1, wherein the shell is reusable while the mask member is disposable.

23. (canceled)

24. The apparatus according to claim 1, wherein the shell is detachably attachable to the mask member and easily detachable from the mask member.

25. The apparatus according to claim 1, wherein the shell is cup-shaped.

26. The apparatus according to claim 1, wherein the shell comprises one or more outlets for guiding away gas escaped from the mask member and captured by the shell.

27. (canceled)

28. The apparatus according to claim 1, wherein the coupler is connectable to a cylinder configured to contain a gas or a gas mixture.

29. The apparatus according to claim 1, wherein the coupler is configured to feed a gas mixture to the mask member, the gas mixture comprising or consisting of nitrous oxide and oxygen.

30. (canceled)

31. (canceled)

32. (canceled)

33. (canceled)

34. (canceled)

35. (canceled)

36. (canceled)

37. (canceled)

38. (canceled)

39. (canceled)
